(19) United States
(12) Patent Application Publication
(10) Pub. No.: US 2026/0224282 A1
(43) Pub. Date: Aug. 6, 2026

(54) MEDICAL DEVICE HAVING EXPANSION BODY

(71) Applicant: TERUMO KABUSHIKI KAISHA, Tokyo (JP)

(72) Inventor: Yusuke TAKAHASHI, Hadano-shi (JP)

(73) Assignee: TERUMO KABUSHIKI KAISHA, Tokyo (JP)

(21) Appl. No.: 19/629,267

(22) Filed: Mar. 26, 2026

Related U.S. Application Data
(63) Continuation of application No. PCT/JP2024/032325, filed on Sep. 10, 2024.

(30) Foreign Application Priority Data
Sep. 26, 2023 (JP) 2023-163537

(51) Int. Cl. A61B 18/14; A61B 18/00

(52) U.S. Cl. CPC A61B 18/1492; A61B 2018/00172; A61B 2018/00184; A61B 2018/00267; A61B 2018/1475

(57) ABSTRACT

A medical device includes: an expandable expansion body including multiple linear bodies; a shaft portion connected to a proximal part of the expansion body; and a pulling portion movable along an axial direction of the shaft portion. The expansion body includes a recess recessed radially inward in an expanded state and including a bottom. The shaft portion includes a hollow distal extension portion extending toward a distal part of the expansion body along a central axis inside the expansion body. The distal extension portion has a distal end located on a distal side with respect to the bottom of the expansion body in the expanded state and on a proximal side with respect to the distal part of the expansion body. The distal extension portion contacts an inner surface of the expansion body between a proximal-side top portion and the distal end of the bottom in a contracted state.

This application is a continuation of International Patent Application No. PCT/JP2024/032325 filed on September 10, 2024, which claims priority to Japanese Patent Application No. 2023-163537 filed on September 26, 2023, the entire content of both of which is incorporated herein by reference.

### TECHNOLOGICAL FIELD

The present invention relates generally to a medical device in which an expansion body that can be expanded and contracted has a recess.

### BACKGROUND DISCUSSION

A known device for performing an ablation treatment to cauterize biological tissue by a high-frequency current includes an expansion body configured to be expanded and contracted in a living body, and on which is disposed an electrode unit. One such treatment by ablation involves a shunt treatment on the atrial septum. Such a shunt treatment can alleviate heart failure symptoms of a patient with heart failure by forming a shunt (through hole) serving as an escape route for an increased atrial pressure in the fossa ovalis of the atrial septum of the patient. In this shunt treatment, the atrial septum is accessed using an intravenous approaching method, and a shunt with a desired size is formed.

WO 2019-085841 discloses an expansion body that has a recess that is recessed radially inward during expansion of the expansion body to define a reception space capable of receiving the biological tissue. The electrode unit is disposed in the recess. In addition, a shaft portion having the expansion body at the distal part includes a pulling portion capable of pulling the expansion body so as to change the shape and the radial position of the recess. The recess is deformed by the pulling portion, and thus, can hold the biological tissue received in the reception space from both sides in the thickness direction.

### SUMMARY

The expansion body of such devices includes linear bodies and can expand and contract radially, with its natural state, where no force is received from the outside, being an expanded state. During the insertion of the medical device, the expansion body is stored and contracted in a storage sheath. When being inserted up to a site to be treated, the storage sheath is moved to a proximal side, by which the expansion body can be exposed and expanded.

In a case where a force in the direction of twist is applied to the expansion body before the expansion body is exposed to the outside of the storage sheath, such as during the insertion of the medical device, a section to become a recess of the expansion body may be twisted and cause entanglement of the linear bodies that form the recess, so that the expansion body cannot be expanded to an expected shape when the expansion body is thereafter expanded. In addition, when the expansion body is twisted inside the storage sheath, the linear bodies forming the expansion body are rubbed against each other, so that coatings applied to the linear bodies may be peeled off or an electrode unit disposed on the linear bodies may be damaged.

A medical device disclosed here is capable of preventing a section to become a recess of an expansion body from being twisted due to displacement in a circumferential direction even when a force in a direction of twist is applied at the time of contraction of the expansion body.

An expansion body disclosed here includes a plurality of linear bodies, including a central axis, and expandable in a radial direction from a contracted state to an expanded state by a self-expanding force; an elongated hollow shaft portion that is connected to a proximal part of the expansion body; and an elongated pulling portion, the pulling portion being inserted into the shaft portion and movable along an axial direction of the shaft portion, wherein the expansion body includes a recess that is recessed radially inward in the expanded state, the recess includes a bottom positioned on an innermost side in the radial direction, a proximal-side upright portion extending from a proximal end of the bottom toward a proximal-side top portion on a radially outer side, and a distal-side upright portion extending from a distal end of the bottom toward a distal-side top portion on a radially outer side, the shaft portion includes a connection portion connected to the proximal part of the expansion body, and a distal extension portion that is hollow and that extends from the connection portion toward a distal part of the expansion body along the central axis inside the expansion body, the pulling portion is inserted into the distal extension portion, the pulling portion being exposed to an outside from the distal extension portion and extending to be connectable to the expansion body so as to apply a pulling force to change a shape and a radial position of the recess to the expansion body, the distal extension portion has a distal end that is located on a distal side with respect to the bottom of the expansion body in the expanded state and is located on a proximal side with respect to the distal part of the expansion body, and the distal extension portion is in contact with an inner surface of the expansion body between the proximal-side top portion and the distal end of the bottom in the contracted state.

In the medical device configured as described above, the distal extension portion extends so as to be in contact with the inner surface of the expansion body between the proximal-side top portion and the distal end of the bottom in the contracted state. Thus, when a force in the direction of twist is applied to the expansion body in the contracted state, the displacement of the linear bodies forming the expansion body in the circumferential direction is restricted, whereby the twist of the expansion body at the recess can be inhibited. Therefore, the medical device can prevent the expansion body from being incapable of expanding to the expected shape, prevent coatings on the linear bodies from being peeled off, and prevent an electrode unit from being damaged.

In the medical device, the expansion body may include a distal-side inclined portion extending from the distal-side top portion toward the distal part of the expansion body, and the distal part of the expansion body may include a convergence portion in which the plurality of linear bodies constituting the distal-side inclined portion is converged. Accordingly, in the medical device, in a case where the expansion body has a shape that is likely to be twisted, the twist at the recess can be inhibited by the distal extension portion.

The medical device may be used together with a sheath into which the shaft portion is insertable, the sheath being capable of storing the expansion body in such a manner that the expansion body is in the contracted state, and when the expansion body is stored in the sheath and is in the contracted state, the proximal-side top portion and the distal-side top portion may be in contact with an inner surface of the sheath, and the bottom may be separated from the inner surface of the sheath. Accordingly, in the medical device, displacement of the linear bodies in the circumferential direction can be restricted in a state where the expansion body is stored in the sheath, whereby the twist of the expansion body at the recess can be inhibited.

In the medical device, the distal end of the distal extension portion may be located on a proximal side with respect to the bottom of the expansion body in the contracted state and on a distal side with respect to the proximal-side top portion of the expansion body, and may be in contact with an inner surface of a section of the proximal-side upright portion in which an outer diameter of the expansion body gradually decreases toward the bottom in the contracted state. Accordingly, in the medical device, the shaft portion is not located inside the bottom located on the innermost part of the expansion body in the radial direction. Thus, the expansion body is easily contracted, whereby the storage of the expansion body can be facilitated.

In the medical device, the distal end of the distal extension portion may be located immediately below an innermost position of the bottom of the expansion body in the radial direction in the contracted state, and may be in contact with an inner surface of the bottom of the expansion body. Accordingly, in the medical device, the displacement of the linear bodies forming the recess in the circumferential direction can be further restricted by the distal extension portion, and thus, the twist of the expansion body can be effectively inhibited.

In the medical device, the pulling portion may include a pulling shaft that is inserted into the shaft portion and that is movable along an axial direction of the shaft portion, and the pulling shaft may be exposed from an interior of the distal extension portion to an outside, extend to the distal side beyond a distal end of the expansion body, and move relative to the shaft portion in a proximal direction in such a manner as to be connected to the distal part of the expansion body to compress the expansion body in the axial direction and to apply a pulling force that changes a shape and a radial position of the recess to the expansion body. Accordingly, in the medical device having a structure in which the distal part of the expansion body is pulled to the proximal side by the pulling shaft to compress the expansion body and to hold the biological tissue in the recess, the twist of the expansion body can be reliably inhibited.

In the medical device, the expansion body may include a distal rigid portion extending from the distal part of the expansion body toward an interior of the expansion body, the distal extension portion and the distal rigid portion may be both more rigid than the pulling shaft, in the expanded state, the pulling shaft may be externally exposed between the distal extension portion and the distal rigid portion, and may be connected to the distal part of the expansion body to pull the distal rigid portion toward the distal extension portion in such a manner that the shape and radial position of the recess are changed, and in the expanded state, a distance between the proximal-side top portion and the distal-side top portion in the axial direction along the central axis may be substantially equal to a length along the axial direction of the pulling shaft exposed between the distal extension portion and the distal rigid portion. Accordingly, in the medical device, when the proximal-side top portion and the distal-side top portion come into contact with each other by pulling the pulling shaft, the rigid distal extension portion and the distal rigid portion come into contact with each other, so that further pulling is restricted, and breakage of the expansion body due to strong contact between the proximal-side top portion and the distal-side top portion can be prevented.

### BRIEF DESCRIPTION OF THE DRAWINGS

FIG. 1 is a front view illustrating an overall configuration of a medical device according to an embodiment;

FIG. 2 is an enlarged perspective view illustrating the vicinity of an expansion body of the medical device;

FIG. 3 is an enlarged front view of the vicinity of the expansion body of the medical device;

FIG. 4 is an enlarged front view of the vicinity of a distal part of the medical device in a state where the expansion body is stored in a storage sheath;

FIG. 5 is an enlarged front view of the vicinity of the distal part of the medical device in a state where the expansion body is stored in the storage sheath in a case where a distal end position of the shaft portion is at the position of a bottom of a recess of the contracted expansion body;

FIG. 6 is an explanatory diagram schematically illustrating a state where the expansion body is placed in an atrial septum, and illustrates a front view of the medical device and a cross-sectional view of a biological tissue;

FIG. 7 is an enlarged front view of the vicinity of the distal part of the medical device in a state where a distal side of the expansion body placed in a through hole is exposed from the storage sheath; and

FIG. 8 is an enlarged front view illustrating a state in which the expansion body grips a biological tissue.

### DETAILED DESCRIPTION

Hereinafter, an embodiment of the medical device having an expansion body, representing examples of the new medical device having an expansion body disclosed here will be described with reference to the drawings. Note that dimensional ratios in the drawings may be exaggerated and different from actual ratios for convenience of description. In addition, in the present specification, a side of a medical device 10 that is to be inserted into a biological lumen will be referred to as a "distal end" or a "distal side", and a side near the operator's hand operating the medical device 10 will be referred to as a "proximal end" or a "proximal side".

The medical device according to the embodiment described below is configured to expand a through hole Hh formed in an atrial septum HA of the heart H of a patient, and to further perform a maintenance procedure to maintain the expanded through hole Hh at the increased size.

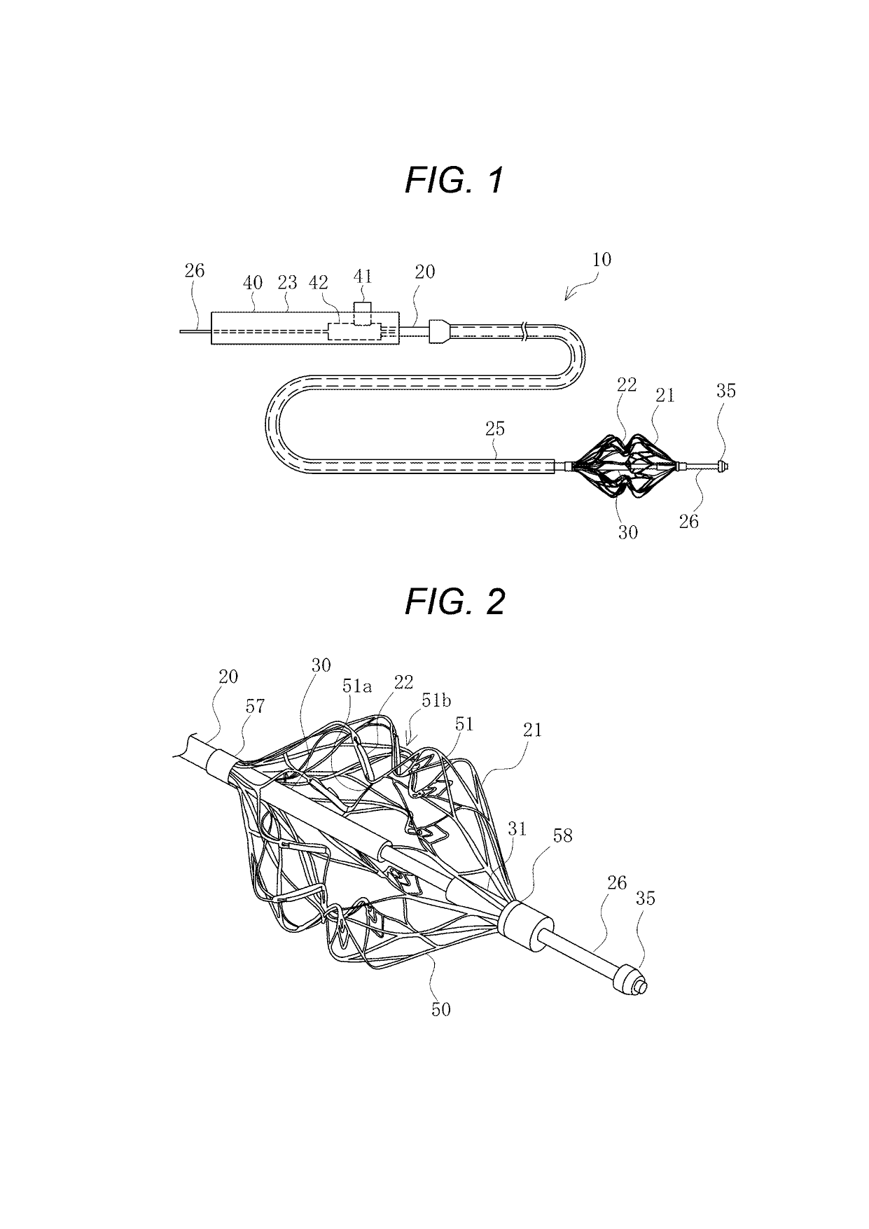

As illustrated in FIGS. 1 and 2, the medical device 10 according to the present embodiment includes an elongated hollow shaft portion 20, an expansion body 21 disposed on a distal part of the shaft portion 20, and a manual operation unit 23 disposed on a proximal part of the shaft portion 20. The expansion body 21 has an electrode unit 22 which is an energy transfer element for performing the above-described maintenance procedure. The expansion body 21 is constituted by linear bodies 50, and is in a state of being expanded in a radial direction due to a self-expansion force in a natural state where no force is received from the outside.

The shaft portion 20 includes a storage sheath 25 disposed on an outermost peripheral portion. The expansion body 21 is movable forward and rearward in an axial direction with respect to the storage sheath 25. The storage sheath 25 can store the expansion body 21 therein when moved to the distal side of the shaft portion 20. The expansion body 21 stored in the storage sheath 25 is in a contracted state. The expansion body 21 can be exposed from the storage sheath 25 and expanded by moving the storage sheath 25 that has stored the expansion body 21 to the proximal side.

A pulling shaft 26 serving as a pulling portion is disposed in the shaft portion 20 so as to be slidable with respect to the shaft portion 20. The pulling shaft 26 is disposed from a position proximal of the manual operation unit 23 to a position distal of the expansion body 21. The pulling shaft 26 protrudes from the distal part of the shaft portion 20, passes through the inside of the expansion body 21, and protrudes from the distal end of the expansion body 21. A distal part of the pulling shaft 26 is fixed to a distal end member 35.

The distal end member 35 to which the distal part of the pulling shaft 26 is fixed is not fixed to the expansion body 21. As a result, when the pulling shaft 26 slides in a proximal direction with respect to the shaft portion 20 along the axial direction, the distal end member 35 can apply a compressive force to the expansion body 21 along the axis of the shaft portion 20. In addition, when the expansion body 21 is stored in the storage sheath 25, the distal end member 35 is moved away from the expansion body 21 to the distal side, by which the expansion body 21 can be rather easily moved in a direction of extension of the expansion body 21, and thus, storage capability can be improved.

The manual operation unit 23 has a housing 40 to be held by an operator, an operation dial 41 that can be rotationally operated by the operator, and a conversion mechanism 42 operated in conjunction with the rotation of the operation dial 41. The pulling shaft 26 is held by the conversion mechanism 42 inside the manual operation unit 23. The conversion mechanism 42 can move the held pulling shaft 26 forward and rearward in the axial direction in conjunction with the rotation of the operation dial 41. For example, a rack and pinion mechanism can be used as the conversion mechanism 42.

It is preferable that the shaft portion 20 is formed of a material having a certain degree of flexibility. Examples of such a material include polyolefin such as polyethylene, polypropylene, polybutene, ethylene-propylene copolymer, ethylene-vinyl acetate copolymer, ionomer, or a mixture of two or more of them, soft polyvinyl chloride resin, polyamide, polyamide elastomer, polyester, polyester elastomer, polyurethane, fluorine resin such as polytetrafluoroethylene, polyimide, PEEK, silicone rubber, and latex rubber.

The pulling shaft 26 can be formed of, for example, an elongated linear body including a super elasticity alloy such as a nickel-titanium alloy and a copper-zinc alloy, a metal material such as stainless steel, a resin material having comparatively high rigidity, or the like.

The distal end member 35 can be formed of, for example, a super elasticity alloy such as a nickel-titanium alloy or a copper-zinc alloy, a metal material such as stainless steel, a polymer material such as polyolefin, polyvinyl chloride, polyamide, polyamide elastomer, polyurethane, polyurethane elastomer, polyimide, or fluorine resin, or a mixture of the above polymer materials. Alternatively, the distal end member 35 can be formed of a multilayer tube containing two or more kinds of polymer materials.

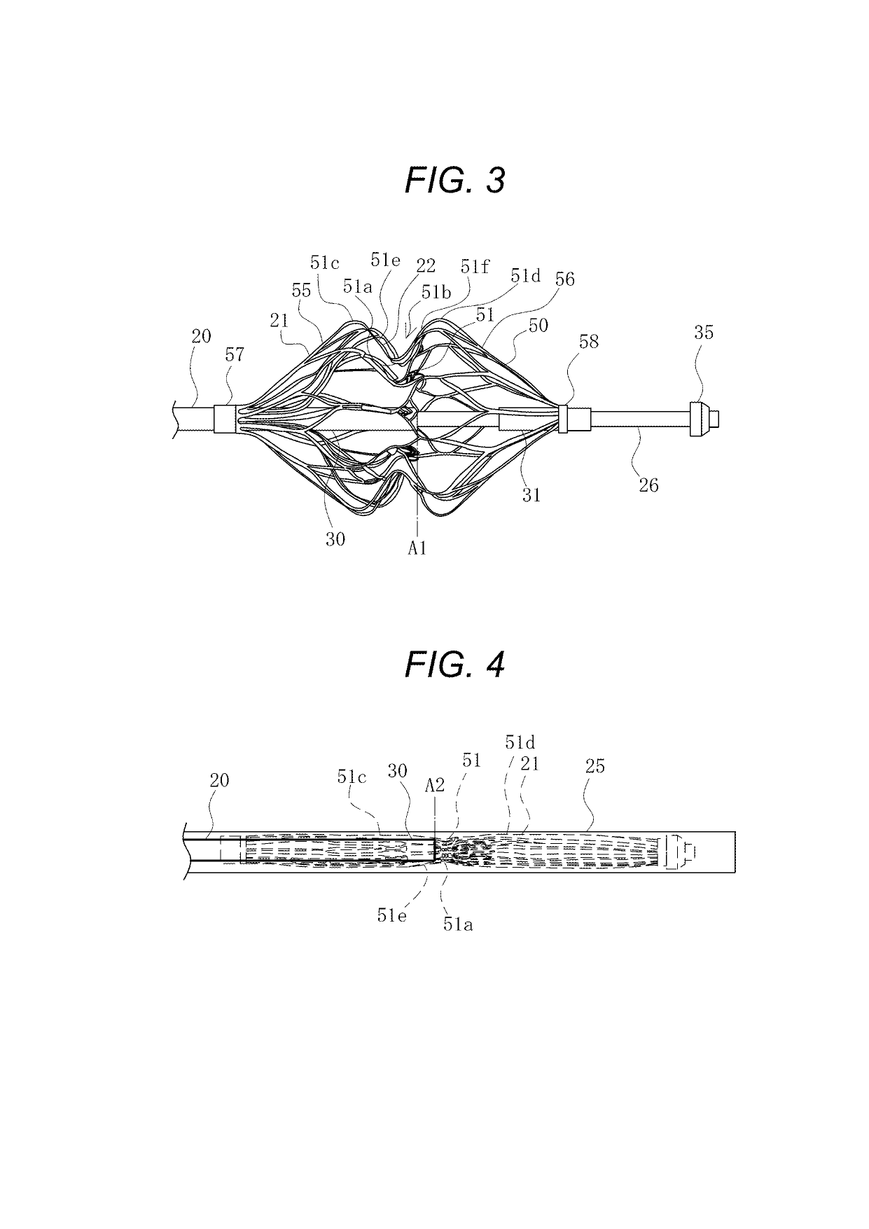

As illustrated in FIG. 3, the shaft portion 20 includes, at the distal part, a hollow distal extension portion 30 extending to the interior of the expansion body 21. The distal extension portion 30 extends from the proximal part of the expansion body 21 to the middle of the expansion body 21 along the central axis of the expansion body 21. In a state where the expansion body 21 is expanded in the radial direction, a distal end position A1 of the distal extension portion 30 in the major axis direction is located on a distal side with respect to a bottom 51a of a recess 51. In addition, the shaft portion 20 has a connection portion 57 connected to the proximal part of the expansion body 21.

The pulling shaft 26 is inserted into the distal extension portion 30 constituting the shaft portion 20, protrudes from the distal end of the distal extension portion 30, and extends through the inside of the expansion body 21 to the distal end member 35 from the distal end of the expansion body 21 so as to apply a pulling force to change the shape and the radial position of the recess 51 to the expansion body 21. The distal part of the pulling shaft 26 is fixed to the distal end member 35.

The distal part of the expansion body 21 is provided with a distal rigid portion 31 extending from the distal part toward the interior of the expansion body 21. The distal extension portion 30 and the distal rigid portion 31 are both more rigid than the pulling shaft 26, which is comparatively soft. The pulling shaft 26 passes through the interior of the distal extension portion 30 and the distal rigid portion 31, and the section between the distal extension portion 30 and the distal rigid portion 31 is exposed to the outside. The expansion body 21 has the distal rigid portion 31. Therefore, when a biological tissue around the puncture hole Hh varies in thickness in the circumferential direction, the pulling shaft 26 can be bent according to the thickness of the biological tissue, and the recess 51 of the expansion body 21 can be in close contact with the biological tissue over the entire circumference in the circumferential direction. In other words, the flexibility of the pulling shaft 26 relative to the distal extension portion 30 and the distal rigid portion 31 enables it to be bent to accommodate non-uniformity of the biological tissue defining the puncture hole Hh in positioning of the recess 51. In addition, in the expansion body 21, the distance between a proximal-side top portion 51c and a distal-side top portion 51d in the axial direction along the central axis is substantially the same as the length of the section of the pulling shaft 26 exposed between the distal extension portion 30 and the distal rigid portion 31 along the axial direction of the pulling shaft 26.

The expansion body 21 includes a plurality of linear bodies 50 in the circumferential direction. The linear bodies 50 form a mesh-shaped structure by branching and joining along a length direction. As a result, the expansion body 21 can expand and contract in a radial direction. Proximal parts of the linear bodies 50 extend to the distal side from the connection portion 57. Distal parts of the linear bodies 50 extend to the proximal side from a distal convergence portion 58. In a state where the expansion body 21 is expanded, the linear bodies 50 include a proximal-side inclined portion 55 inclined so as to increase in the radial direction from the connection portion 57 toward the central part, and a distal-side inclined portion 56 inclined so as to increase in the radial direction from the distal convergence portion 58 toward the central part.

The linear bodies 50 have a recess 51 recessed inward in the radial direction of the expansion body 21 in the expanded state at the central part in the axial direction. An innermost part of the recess 51 in the radial direction is a bottom 51a. The recess 51 includes a proximal-side upright portion 51e extending to the proximal-side top portion 51c on the radially outer side from the proximal end of the bottom 51a and a distal-side upright portion 51f extending to the distal-side top portion 51d on the radially outer side from the distal end of the bottom 51a. The bottom 51a is a section in which the linear bodies 50 are bent at the innermost side in the radial direction in the direction of extension of the linear bodies 50, and the proximal-side upright portion 51e and the distal-side upright portion 51f are sections in which the linear bodies 50 extend linearly in the direction of extension of the linear bodies 50. The recess 51 defines a reception space 51b that can receive a biological tissue when the expansion body 21 is expanded.

When the pulling shaft 26 slides in the proximal direction with respect to the shaft portion 20 to apply a compressive force to the expansion body 21, the distal-side upright portion 51f and the proximal-side upright portion 51e are brought close to each other, and both portions come in close contact with the biological tissue received in the reception space 51b. The electrode unit 22 is disposed along the recess 51 at the proximal-side upright portion 51e so as to face the reception space 51b. In other words, the electrode unit 22 is disposed along the expansion body 21 in an intermediate part of the expansion body 21 in a central axis direction. In the present embodiment, ten electrode units 22 are disposed along the circumferential direction. The electrode unit 22 may be disposed on the distal-side upright portion 51f.

The linear bodies 50 constituting the expansion body 21 can be formed by, for example, cutting a single metal cylindrical member with laser. The linear bodies 50 can be formed of a metal material. Examples of the metal material that may be used include a titanium-based (Ti-Ni, Ti-Pd, Ti-Nb-Sn, etc.) alloy, a copper-based alloy, stainless steel, β-titanium steel, and a Co-Cr alloy. It is more preferable to use an alloy having spring property such as a nickel titanium alloy. However, the material of the linear bodies 50 is not limited to the above materials, and other materials may be used.

The electrode units 22 are connected to an energy supply device (not illustrated) which is an external device. A high-frequency voltage is applied from the energy supply device to an electrode pair including two electrode units 22, and energy is applied between them. In other words, in the embodiment, the electrode unit 22 is configured as a bipolar electrode. The electrode unit 22 may also be a monopolar electrode. In this case, electric current is supplied between the electrode unit 22 and an external electrode.

As illustrated in FIG. 4, the expansion body 21 stored inside the storage sheath 25 is in a contracted state. In this state, a distal end position A2 in the major axis direction of the distal extension portion 30 constituting the shaft portion 20 is located in the vicinity of the bottom 51a of the expansion body 21. More specifically, the distal end position A2 in the major axis direction of the distal extension portion 30 in a state where the expansion body 21 is contracted is located proximal to the position on the innermost side of the bottom 51a in the radial direction, and is in contact with the inner surface of the expansion body 21 between the proximal-side top portion 51c and the distal end of the bottom 51a. With the distal end position A2 in the major axis direction of the distal extension portion 30 being located in the vicinity of the bottom 51a in a state where the expansion body 21 is contracted, the distal end position A1 in the major axis direction of the distal extension portion 30 will be located distal to the bottom 51a and proximal to the distal part of the expansion body 21 upon expansion of the expansion body 21 as illustrated in FIG. 3. Since the distal end position A1 of the distal extension portion 30 in the major axis direction is located distal to the bottom 51a at the time of expansion of the expansion body 21, part of the distal extension portion 40 extending from the proximal part of the expansion body 21 to the distal side beyond the bottom 51a defines a central shaft inside the expansion body 21. Therefore, the proximal side of the expansion body 21 with respect to the bottom 51a is less likely to be bent, and the electrode units 22 disposed along the proximal-side upright portion 51e can be reliably pressed against the biological tissue.

It is only necessary that the distal end position A2 in the major axis direction of the distal extension portion 30 in a state where the expansion body 21 is contracted is located in the vicinity of the bottom 51a, and, as such, may be located, for example, at an area of the bottom 51a or at an area of the proximal-side upright portion 51e in the direction of extension of the linear bodies 50. The distal end position A2 of the distal extension portion 30 in the major axis direction may also be located at a boundary between the bottom 51a and the proximal-side upright portion 51e in the direction of extension of the linear bodies 50.

The expansion body 21 has a shape in which the linear bodies 50 distal to the recess 51 extend to the distal convergence portion 58, and the distal side is closed. Therefore, when a force acts in a direction of twist between the distal end and the proximal end of the expansion body 21, the linear bodies 50 tend to, at the position of the recess 51, be twisted and caused to collapse inward, thus rubbing against each other. In the medical device 10 according to the present embodiment, the distal end position A2 in the major axis direction of the distal extension portion 30 in a state where the expansion body 21 is contracted is located in the vicinity of the bottom 51a of the expansion body 21, so that the distal extension portion 30 extends to the vicinity of the bottom 51a. The distal extension portion 30 in this position can thus act as a stopper to prevent the linear bodies 50 from twisting and collapsing inward at the position of the recess 51. In addition, since the twist of the linear bodies 50 can be inhibited, the rubbing between the linear bodies 50 can be inhibited, and peeling of coatings applied to the surfaces of the linear bodies 50 and breakage of the electrode units 22 can be prevented.

When the expansion body 21 is stored in the storage sheath 25 to be in a stored state, the proximal-side top portion 51c and the distal-side top portion 51d are in contact with the inner surface of the storage sheath 25, and the bottom 51a is separated from the inner surface of the storage sheath 25. At this time, the distal end of the distal extension portion 30 comes into contact with the inner surface of a section of the proximal-side upright portion 51c where the outer diameter of the expansion body 21 gradually decreases toward the bottom 51a. That is, the shaft portion 20 is not located inside the bottom 51a located on the innermost part of the expansion body 21 in the radial direction. Therefore, the expansion body 21 can be easily contracted, whereby the expansion body 21 can be easily stored.

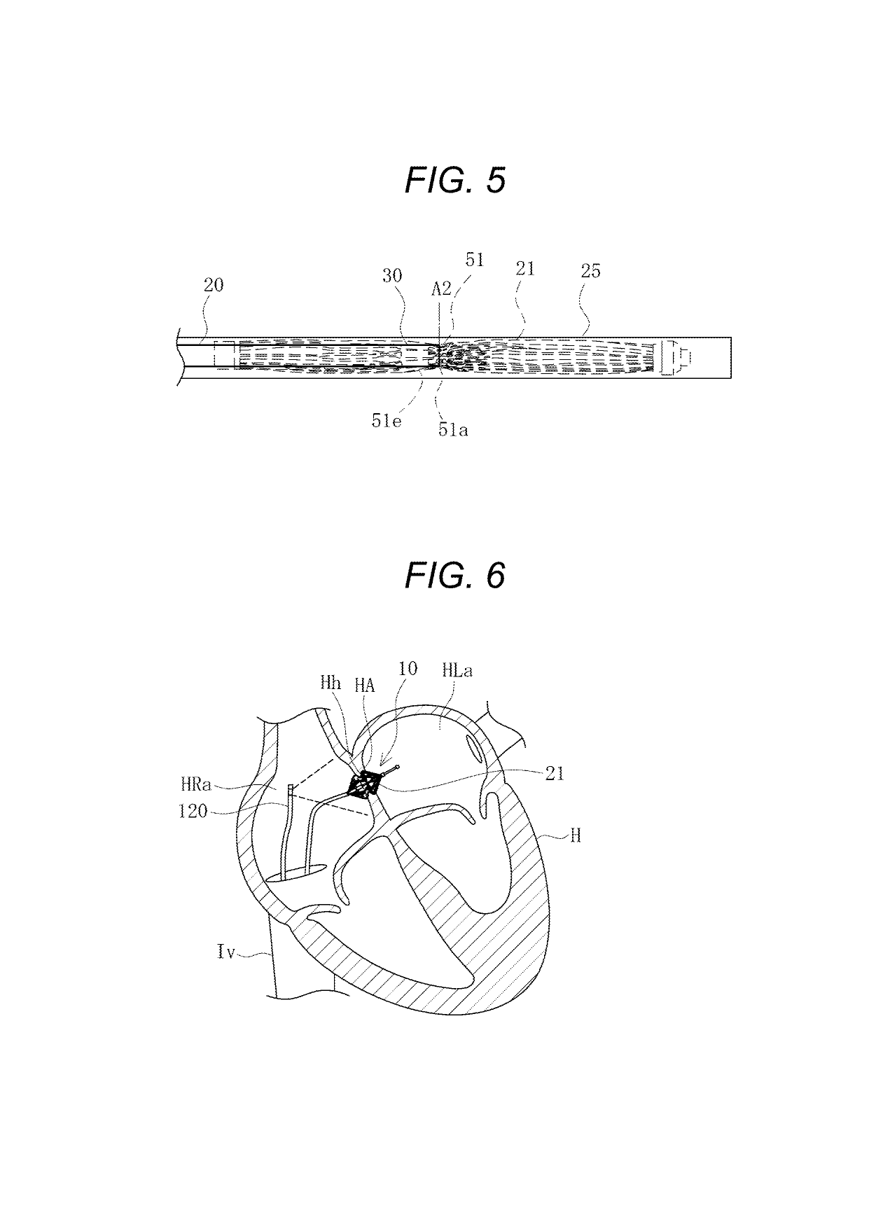

As illustrated in FIG. 5, the distal end position A2 in the major axis direction of the distal extension portion 30 in a state where the expansion body 21 is contracted may be located immediately below the innermost position of the bottom 51a in the radial direction and may be in contact with the inner surface of the bottom 51a of the expansion body 21. As a result, the displacement of the linear bodies 50 in the circumferential direction at the position of the bottom 51a is further restricted, so that the twist of the expansion body 21 can be more effectively inhibited.

When the medical device 10 is used, the through hole Hh is formed in advance at the position of the fossa ovalis of the atrial septum HA using a puncture device. The medical device 10 performs a procedure of expanding the through hole Hh and cauterizing an edge of the through hole Hh to maintain the expanded through hole Hh at its increased size. As illustrated in FIG. 4, the medical device 10 is delivered from an inferior vena cava Iv to the vicinity of the atrial septum HA via a right atrium HRa, and the storage sheath 25 is inserted to the atrial septum HA so that the expansion body 21 is placed at the position of the through hole Hh that has been formed in advance. The distal part of the storage sheath 25 penetrates the atrial septum HA to reach the left atrium HLa.

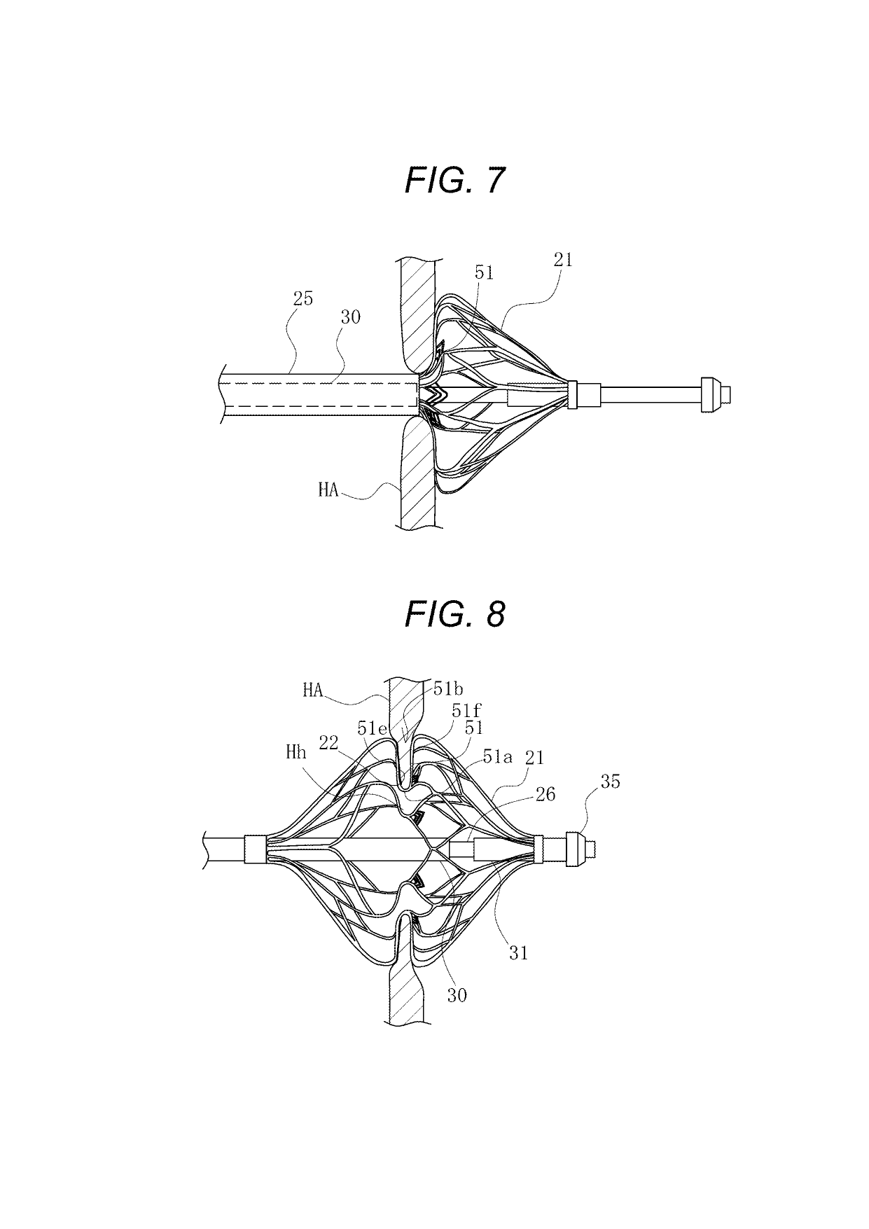

During the insertion of the medical device 10, the expansion body 21 is stored and contracted in the storage sheath 25. After the storage sheath 25 penetrates the atrial septum HA, the storage sheath 25 is moved to the proximal side, by which the expansion body 21 can be expanded such that the section of the expansion body 21 distal to the recess 51 is exposed as illustrated in FIG. 7. Even if a force in a direction of twist is applied to the expansion body 21 during the insertion of the medical device 10, the twist of the expansion body 21 is inhibited due to the distal extension portion 30 extending up to the vicinity of the bottom 51a of the recess 51 as described above, and thus, a failure of expansion due to the twist of the expansion body 21 can be prevented.

When the storage sheath 25 is further moved to the proximal side to expose the entire expansion body 21, a section of the expansion body 21 proximal to the recess 51 is also expanded in the radial direction, and the recess 51 is placed in the through hole Hh of the atrial septum HA and receives the biological tissue surrounding the through hole Hh in the reception space 51b.

The pulling shaft 26 is moved to the proximal side in a state in which the reception space 51b receives the biological tissue, by which the expansion body 21 is pulled in a compression direction by the distal end member 35 to be compressed in the axial direction, the atrial septum HA is gripped by the proximal-side upright portion 51e and the distal-side upright portion 51f which constitute the recess 51, and the electrode units 22 are pressed against the biological tissue, as illustrated in FIG. 8. The fossa ovalis where the through hole Hh is formed has a smaller wall thickness than other parts of the atrial septum HA. Therefore, the recess 51 of the expansion body 21 can grip the biological tissue so as to crush the biological tissue. At this time, the distal end of the distal extension portion 30 and the proximal end of the distal rigid portion 31 come close to each other. The distal end position of the distal extension portion 30 in the major axis direction is set so as not to be positioned distal to the bottom 51a in a contracted state of the expansion body 21 in order that the distal end does not interfere with the proximal end of the distal rigid portion 31 in a state where the biological tissue is gripped by the expansion body 21.

In a state where the electrode units 22 are pressed against the biological tissue, high frequency energy is applied to the edge of the through hole Hh through the electrode units 22, whereby the edge of the through hole Hh can be cauterized (heated and cauterized) by the high frequency energy. The high frequency energy is applied by applying a voltage between the pair of electrode units 22 adjacent to each other in the circumferential direction. This results in making it possible to inhibit blockage of the through hole Hh due to natural healing and maintain a size thereof.

When the medical device 10 is used, hemodynamics is checked by a hemodynamics checking device 120 delivered to the right atrium HRa via the inferior vena cava Iv. As the hemodynamics checking device 120, a known echo catheter can be used, for example. The operator can display an echo image obtained by the hemodynamics checking device 120 on a display device, such as a display, and can check the volume of blood passing through the through hole Hh on the basis of a displayed result.

As described above, (1) a medical device 10 according to the present embodiment includes: an expansion body 21 including a plurality of linear bodies 50, including a central axis, and expandable in a radial direction from a contracted state to an expanded state by a self-expanding force; an elongated hollow shaft portion 20 that is connected to a proximal part of the expansion body 21; and an elongated pulling portion 26, the pulling portion 26 being inserted into the shaft portion 20 and movable along an axial direction of the shaft portion 20, wherein the expansion body 21 includes a recess 51 that is recessed radially inward in the expanded state, the recess 51 includes a bottom 51a positioned on an innermost side in the radial direction, a proximal-side upright portion 51e extending from a proximal end of the bottom 51a toward a proximal-side top portion 51c on a radially outer side, and a distal-side upright portion 51f extending from a distal end of the bottom 51a toward a distal-side top portion 51d on a radially outer side, the shaft portion 20 includes a connection portion 57 connected to the proximal part of the expansion body 21, and a distal extension portion 30 that is hollow and that extends from the connection portion 57 toward a distal part of the expansion body 21 along the central axis inside the expansion body 21, the pulling portion 26 is inserted into the distal extension portion 30, the pulling portion 26 being exposed to an outside from the distal extension portion 30 and extending to be connectable to the expansion body 21 so as to apply a pulling force to change a shape and a radial position of the recess 51 to the expansion body 21, the distal extension portion 30 includes a distal end that is located on a distal side with respect to the bottom 51a of the expansion body 21 in the expanded state and is located on a proximal side with respect to the distal part of the expansion body 21, and the distal extension portion 30 is in contact with an inner surface of the expansion body 21 between the proximal-side top portion 51c and the distal end of the bottom 51a in the contracted state. In the medical device 10 configured as described above, the distal extension portion 30 extends so as to be in contact with the inner surface of the expansion body 21 between the proximal-side top portion 51c and the distal end of the bottom 51a in the contracted state. Thus, when a force in the direction of twist is applied to the expansion body 21 in the contracted state, the displacement of the linear bodies 50 forming the expansion body 21 in the circumferential direction is restricted, whereby the twist of the expansion body 21 at the recess 51 can be inhibited. Therefore, the medical device 10 can prevent the expansion body 21 from being incapable of expanding to the expected shape, prevent coatings on the linear bodies 50 from being peeled off, and prevent an electrode unit from being damaged.

(2) In the medical device 10 according to (1), the expansion body 21 may include a distal-side inclined portion 56 extending from the distal-side top portion 51d toward the distal part of the expansion body 21, and the distal part of the expansion body 21 may include a distal convergence portion 58 in which the plurality of linear bodies 50 constituting the distal-side inclined portion 56 is converged. Accordingly, in the medical device 10, in a case where the expansion body 21 has a shape that is likely to be twisted, the twist in the recess 51 can be inhibited by the distal extension portion 30.

(3) The medical device 10 according to (1) or (2) may be used together with a sheath 25 into which the shaft portion 20 is insertable, the sheath 25 being capable of storing the expansion body 21 in such a manner that the expansion body 21 is in the contracted state, and when the expansion body 21 is stored in the sheath 25 and is in the contracted state, the proximal-side top portion 51c and the distal-side top portion 51d may be in contact with an inner surface of the sheath 25, and the bottom 51a may be separated from the inner surface of the sheath 25. Accordingly, in the medical device 10, displacement of the linear bodies 50 in the circumferential direction can be restricted in a state where the expansion body 21 is stored in the sheath 25, whereby the twist of the expansion body 21 at the recess 51 can be inhibited.

(4) In the medical device 10 according to any one of (1) to (3), the distal end of the distal extension portion 30 may be located on a proximal side with respect to the bottom 51a of the expansion body 21 in the contracted state and on a distal side with respect to the proximal-side top portion 51c of the expansion body 21, and may be in contact with an inner surface of a section of the proximal-side upright portion 51c in which an outer diameter of the expansion body 21 gradually decreases toward the bottom 51a in the contracted state. Accordingly, in the medical device 10, the shaft portion 20 is not located inside the bottom 51a located on the innermost part of the expansion body 21 in the radial direction. Thus, the expansion body 21 is easily contracted, whereby the storage of the expansion body 21 can be facilitated.

(5) In the medical device 10 according to any one of (1) to (4), the distal end of the distal extension portion 30 may be located immediately below an innermost position of the bottom 51a of the expansion body 21 in the radial direction in the contracted state, and may be in contact with an inner surface of the bottom 51a of the expansion body 21. Accordingly, in the medical device 10, the displacement of the linear bodies 50 forming the recess 51 in the circumferential direction can be further restricted by the distal extension portion 30, and thus, the twist of the expansion body 21 can be effectively inhibited.

(6) In the medical device 10 according to any one of (1) to (5), the pulling portion 26 may include a pulling shaft 26 that is inserted into the shaft portion 20 and that is movable along an axial direction of the shaft portion 20, and the pulling shaft 26 may be exposed from an interior of the distal extension portion 30 to an outside, extend to the distal side beyond a distal end of the expansion body 21, and move relative to the shaft portion 20 in a proximal direction in such a manner as to be connected to the distal part of the expansion body 21 to compress the expansion body 21 in the axial direction and to apply a pulling force that changes a shape and a radial position of the recess 51 to the expansion body 21. Accordingly, in the medical device 10 having a structure in which the distal part of the expansion body 21 is pulled to the proximal side by the pulling shaft 26 to compress the expansion body 21 and to hold the biological tissue in the recess 51, the twist of the expansion body 21 can be reliably inhibited.

(7) In the medical device 10 according to (6), the expansion body 21 may include a distal rigid portion 31 extending from the distal part of the expansion body 21 toward an interior of the expansion body 21, the distal extension portion 30 and the distal rigid portion 31 may be both more rigid than the pulling shaft 26, in the expanded state, the pulling shaft 26 may be externally exposed between the distal extension portion 30 and the distal rigid portion 31, and may be connected to the distal part of the expansion body 21 to pull the distal rigid portion 31 toward the distal extension portion 30 in such a manner that the shape and radial position of the recess 51 are changed, and in the expanded state, a distance between the proximal-side top portion 51c and the distal-side top portion 51d in the axial direction along the central axis may be substantially equal to a length along the axial direction of the pulling shaft 26 exposed between the distal extension portion 30 and the distal rigid portion 31. Accordingly, in the medical device 10, when the proximal-side top portion 51c and the distal-side top portion 51d come into contact with each other by pulling the pulling shaft 26, the rigid distal extension portion 30 and the distal rigid portion 31 come into contact with each other, so that further pulling is restricted, and breakage of the expansion body 21 due to strong contact between the proximal-side top portion 51c and the distal-side top portion 51d can be prevented.

The detailed description above describes embodiments of a medical device having an expansion body representing examples of the new medical device having

an expansion body disclosed here. The invention is not limited, however, to the precise embodiment and modifications described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims. For example, in the above-described embodiments, the pulling portion is the pulling shaft 26, but the pulling portion may be a mechanism other than the pulling shaft 26, and may be, for example, a wire or the like connected to the bottom 51a of the recess 51.

What is claimed is:

1. A medical device comprising:
an expansion body including a plurality of linear bodies, including a central axis, and expandable in a radial direction from a contracted state to an expanded state by a self-expanding force;
an elongated hollow shaft portion that is connected to a proximal part of the expansion body; and
an elongated pulling portion, the pulling portion being inserted into the shaft portion and movable along an axial direction of the shaft portion, wherein
the expansion body includes a recess that is recessed radially inward in the expanded state,
the recess includes a bottom positioned on an innermost side in the radial direction, a proximal-side upright portion extending from a proximal end of the bottom toward a proximal-side top portion on a radially outer side, and a distal-side upright portion extending from a distal end of the bottom toward a distal-side top portion on a radially outer side,
the shaft portion includes a connection portion connected to the proximal part of the expansion body, and a distal extension portion that is hollow and that extends from the connection portion toward a distal part of the expansion body along the central axis inside the expansion body,
the pulling portion is inserted into the distal extension portion, the pulling portion being exposed to an outside from the distal extension portion and extending to be connectable to the expansion body so as to apply a pulling force to the expansion body to change a shape and a radial position of the recess,
the distal extension portion has a distal end that is located on a distal side with respect to the bottom of the expansion body in the expanded state and is located on a proximal side with respect to the distal part of the expansion body, and
the distal extension portion is in contact with an inner surface of the expansion body between the proximal-side top portion and the distal end of the bottom in the contracted state.

2. The medical device according to claim 1, wherein
the expansion body includes a distal-side inclined portion extending from the distal-side top portion toward the distal part of the expansion body, and
the distal part of the expansion body includes a convergence portion in which the plurality of linear bodies constituting the distal-side inclined portion is converged.

3. The medical device according to claim 1,
the medical device being used together with a sheath into which the shaft portion is insertable, the sheath being capable of storing the expansion body in such a manner that the expansion body is in the contracted state, wherein
when the expansion body is stored in the sheath and is in the contracted state, the proximal-side top portion and the distal-side top portion are in contact with an inner surface of the sheath, and the bottom is separated from the inner surface of the sheath.

4. The medical device according to claim 1, wherein the distal end of the distal extension portion is located on a proximal side with respect to the bottom of the expansion body in the contracted state and on a distal side with respect to the proximal-side top portion of the expansion body, and is in contact with an inner surface of a section of the proximal-side upright portion in which an outer diameter of the expansion body gradually decreases toward the bottom in the contracted state.

5. The medical device according to claim 1, wherein the distal end of the distal extension portion is located immediately below an innermost position of the bottom of the expansion body in the radial direction in the contracted state, and is in contact with an inner surface of the bottom of the expansion body.

6. The medical device according to claim 1, wherein
the pulling portion includes a pulling shaft that is inserted into the shaft portion and that is movable along an axial direction of the shaft portion, and
the pulling shaft is exposed from an interior of the distal extension portion to an outside, extends to the distal side beyond a distal end of the expansion body, and moves relative to the shaft portion in a proximal direction in such a manner as to be connected to the distal part of the expansion body to compress the expansion body in the axial direction and to apply a pulling force to the expansion body that changes a shape and a radial position of the recess.

7. The medical device according to claim 6, wherein
the expansion body includes a distal rigid portion extending from the distal part of the expansion body toward an interior of the expansion body,
the distal extension portion and the distal rigid portion are both more rigid than the pulling shaft,
in the expanded state, the pulling shaft is externally exposed between the distal extension portion and the distal rigid portion, and is connected to the distal part of the expansion body to pull the distal rigid portion toward the distal extension portion in such a manner that the shape and radial position of the recess are changed, and
in the expanded state, a distance between the proximal-side top portion and the distal-side top portion in the axial direction along the central axis is substantially equal to a length along the axial direction of the pulling shaft exposed between the distal extension portion and the distal rigid portion.

8. A medical device comprising:
an expansion body including a plurality of linear bodies, including a central axis, and expandable in a radial direction from a contracted state to an expanded state by a self-expanding force;
an elongated hollow shaft portion that is connected to a proximal part of the expansion body; and
an elongated pulling portion, the pulling portion being inserted into the shaft portion and movable along an axial direction of the shaft portion, wherein
the expansion body includes a recess that is recessed radially inward in the expanded state,
the recess includes a bottom positioned on an innermost side in the radial direction, a proximal-side upright portion extending from a proximal end of the bottom toward a proximal-side top portion on a radially outer side, and a distal-side upright portion extending from a distal end of the bottom toward a distal-side top portion on a radially outer side,
the shaft portion includes a connection portion connected to the proximal part of the expansion body, and a distal extension portion that is hollow and that extends from the connection portion toward a distal part of the expansion body along the central axis inside the expansion body,
the pulling portion is inserted into the distal extension portion, the pulling portion being exposed to an outside from the distal extension portion and extending to be connectable to the expansion body so as to apply a pulling force to the expansion body to change a shape of the recess,
the distal extension portion has a distal end that is located on a distal side with respect to the bottom of the expansion body in the expanded state and is located on a proximal side with respect to the distal part of the expansion body, and
the distal extension portion is in contact with an inner surface of the expansion body between the proximal-side top portion and the distal end of the bottom in the contracted state.

9. The medical device according to claim 8, wherein
the expansion body includes a distal-side inclined portion extending from the distal-side top portion toward the distal part of the expansion body, and
the distal part of the expansion body includes a convergence portion in which the plurality of linear bodies constituting the distal-side inclined portion is converged.

10. The medical device according to claim 8,
the medical device being used together with a sheath into which the shaft portion is insertable, the sheath being capable of storing the expansion body in such a manner that the expansion body is in the contracted state, wherein
when the expansion body is stored in the sheath and is in the contracted state, the proximal-side top portion and the distal-side top portion are in contact with an inner surface of the sheath, and the bottom is separated from the inner surface of the sheath.

11. The medical device according to claim 8, wherein the distal end of the distal extension portion is located on a proximal side with respect to the bottom of the expansion body in the contracted state and on a distal side with respect to the proximal-side top portion of the expansion body, and is in contact with an inner surface of a section of the proximal-side upright portion in which an outer diameter of the expansion body gradually decreases toward the bottom in the contracted state.

12. The medical device according to claim 8, wherein the distal end of the distal extension portion is located immediately below an innermost position of the bottom of the expansion body in the radial direction in the contracted state, and is in contact with an inner surface of the bottom of the expansion body.

13. The medical device according to claim 8, wherein
the pulling portion includes a pulling shaft that is inserted into the shaft portion and that is movable along an axial direction of the shaft portion, and
the pulling shaft is exposed from an interior of the distal extension portion to an outside, extends to the distal side beyond a distal end of the expansion body, and moves relative to the shaft portion in a proximal direction in such a manner as to be connected to the distal part of the expansion body to compress the expansion body in the axial direction and to apply a pulling force to the expansion body that changes a shape of the recess.

14. The medical device according to claim 13, wherein
the expansion body includes a distal rigid portion extending from the distal part of the expansion body toward an interior of the expansion body,
the distal extension portion and the distal rigid portion are both more rigid than the pulling shaft,
in the expanded state, the pulling shaft is externally exposed between the distal extension portion and the distal rigid portion, and is connected to the distal part of the expansion body to pull the distal rigid portion toward the distal extension portion in such a manner that the shape of the recess is changed, and
in the expanded state, a distance between the proximal-side top portion and the distal-side top portion in the axial direction along the central axis is substantially equal to a length along the axial direction of the pulling shaft exposed between the distal extension portion and the distal rigid portion.

15. A medical device comprising:
an expansion body including a plurality of linear bodies, including a central axis, and expandable in a radial direction from a contracted state to an expanded state by a self-expanding force;
an elongated hollow shaft portion that is connected to a proximal part of the expansion body; and
an elongated pulling portion, the pulling portion being inserted into the shaft portion and movable along an axial direction of the shaft portion, wherein
the expansion body includes a recess that is recessed radially inward in the expanded state,
the recess includes a bottom positioned on an innermost side in the radial direction, a proximal-side upright portion extending from a proximal end of the bottom toward a proximal-side top portion on a radially outer side, and a distal-side upright portion extending from a distal end of the bottom toward a distal-side top portion on a radially outer side,
the shaft portion includes a connection portion connected to the proximal part of the expansion body, and a distal extension portion that is hollow and that extends from the connection portion toward a distal part of the expansion body along the central axis inside the expansion body,
the pulling portion is inserted into the distal extension portion, the pulling portion being exposed to an outside from the distal extension portion and extending to be connectable to the expansion body so as to apply a pulling force to the expansion body to change a radial position of the recess,
the distal extension portion has a distal end that is located on a distal side with respect to the bottom of the expansion body in the expanded state and is located on a proximal side with respect to the distal part of the expansion body, and
the distal extension portion is in contact with an inner surface of the expansion body between the proximal-side top portion and the distal end of the bottom in the contracted state.

16. The medical device according to claim 15, wherein
the expansion body includes a distal-side inclined portion extending from the distal-side top portion toward the distal part of the expansion body, and
the distal part of the expansion body includes a convergence portion in which the plurality of linear bodies constituting the distal-side inclined portion is converged.

17. The medical device according to claim 15,
the medical device being used together with a sheath into which the shaft portion is insertable, the sheath being capable of storing the expansion body in such a manner that the expansion body is in the contracted state, wherein
when the expansion body is stored in the sheath and is in the contracted state, the proximal-side top portion and the distal-side top portion are in contact with an inner surface of the sheath, and the bottom is separated from the inner surface of the sheath.

18. The medical device according to claim 15, wherein the distal end of the distal extension portion is located on a proximal side with respect to the bottom of the expansion body in the contracted state and on a distal side with respect to the proximal-side top portion of the expansion body, and is in contact with an inner surface of a section of the proximal-side upright portion in which an outer diameter of the expansion body gradually decreases toward the bottom in the contracted state.

19. The medical device according to claim 15, wherein the distal end of the distal extension portion is located immediately below an innermost position of the bottom of the expansion body in the radial direction in the contracted state, and is in contact with an inner surface of the bottom of the expansion body.

20. The medical device according to claim 15, wherein
the pulling portion includes a pulling shaft that is inserted into the shaft portion and that is movable along an axial direction of the shaft portion, and
the pulling shaft is exposed from an interior of the distal extension portion to an outside, extends to the distal side beyond a distal end of the expansion body, and moves relative to the shaft portion in a proximal direction in such a manner as to be connected to the distal part of the expansion body to compress the expansion body in the axial direction and to apply a pulling force to the expansion body that changes a radial position of the recess.
